(19) United States
(12) Patent Application Publication
(10) Pub. No.: US 2025/0144103 A1
(43) Pub. Date: May 8, 2025

(54) L718 AND/OR L792 MUTANT TREATMENT-RESISTANT EGFR INHIBITOR

(71) Applicant: TAIHO PHARMACEUTICAL CO., LTD., Tokyo (JP)

(72) Inventors: Shinichi HASAKO, TSUKUBA-SHI (JP); Takao UNO, TSUKUBA-SHI (JP)

(73) Assignee: TAIHO PHARMACEUTICAL CO., LTD., Tokyo (JP)

(21) Appl. No.: 19/015,893

(22) Filed: Jan. 10, 2025

Related U.S. Application Data
(62) Division of application No. 17/309,847, filed on Jun. 23, 2021.

(30) Foreign Application Priority Data
Dec. 28, 2018 (JP) 2018-247131

(51) Int. Cl. A61K 31/519; A61P 35/00

(52) U.S. Cl. CPC A61K 31/519; A61P 35/00

(57) ABSTRACT

The present invention provides an antitumor agent for treating a malignant tumor patient expressing EGFR having at least one mutation selected from the group consisting of L718X mutation in exon 18 and L792X mutation in exon 20, wherein X represents an arbitrary amino-acid residue, the antitumor agent comprising (S)-N-(4-amino-6-methyl-5-(quinolin-3-yl)-8,9-dihydropy-rimido[5,4-b]indolizin-8-yl)acrylamide (Compound (A)) or a salt thereof.

### CROSS-REFERENCE TO RELATED APPLICATIONS

The present application is a divisional application of U.S. application Ser. No. 17/309,847, filed on Jun. 23, 2021, which is a 35 U.S.C. § 371 national stage patent application of International patent application PCT/JP2019/051377, filed on Dec. 27, 2019, which is based on and claims the benefits of priority to Japanese Application No. 2018-247131, filed on Dec. 28, 2018. The entire contents of all of the above applications are incorporated herein by reference.

### REFERENCE TO A SEQUENCE LISTING

In accordance with 37 CFR § 1.831-1835 and 37 CFR § 1.77(b)(5), the specification makes reference to a Sequence Listing submitted electronically as a .xml file named “556296US_ST26.xml”. This .xml file was generated on Dec. 27, 2024 and is 3,101 bytes in size. The entire contents of the Sequence Listing are hereby incorporated by reference.

### TECHNICAL FIELD

The present invention relates to an antitumor agent against cancers, comprising an exon 18 and/or exon 20 treatment-resistant mutant epidermal growth factor receptor (hereinafter referred to as “EGFR”).

### BACKGROUND ART

EGFR is a receptor-type tyrosine kinase, exerts its physiological function in normal tissue by being bound to Epidermal Growth Factor (hereinafter also referred to as EGF), which is a ligand, and contributes to growth and apoptosis inhibition in epithelial tissues (NPL 1). Further, somatic mutation of EGFR gene has been known as a cancer-causing gene; for example, EGFR in which codons 746 to 750 in exon 19 are deleted (hereinafter also referred to as “exon 19 deletion mutation”) and EGFR in which leucine encoded by codon 858 in exon 21 is mutated to arginine (hereinafter also referred to as “L858R mutation”) constantly induce EGF-independent kinase activity, and contribute to the growth and survival of cancer cells (NPL 2). These mutations are observed, for example, in 30 to 50% of non-small-cell lung cancer in East Asia. The mutations are also observed in about 10% of non-small-cell lung cancer in Europe and the United States, and are regarded as one of the causes of cancers (NPL 3).

Therefore, research and development of EGFR inhibitor as an antitumor agent have actively been conducted, and introduced into the treatment of various EGFR mutation-positive lung cancers (NPL 2 and NPL 4). Gefitinib, erlotinib, and afatinib have been used as therapeutic agents against exon 19 deletion mutant and L858R mutant EGFR-positive lung cancers. Exon 19 deletion mutation and L858R mutation account for 90% of EGFR mutation. Further, occurrence of acquired resistance in the process of the treatment using these agents has been known, and 50% thereof is caused by resistance mutation EGFR in which codon 790 of exon 20 is changed from threonine to methionine (hereinafter also referred to as “T790M mutation”). To treat lung cancers having this mutation, osimertinib has been used as a therapeutic agent. Therefore, treatments using EGFR inhibitors are in the process of being established for lung cancer patients having major EGFR mutations.

### CITATION LIST

### Patent Literature

PTL 1: WO2015/175632A1
PTL 2: WO2015/025936A1

### Non-Patent Literature

Nat. Rev. Cancer, vol. 6, pp. 803-812 (2006) Nature Medicine, vol. 19, pp. 1389-1400 (2013) Nat. Rev. Cancer, vol. 7, pp. 169-181 (2007) Clin. Cancer Res., vol. 24, pp. 3097-3107 (2018)

### SUMMARY OF INVENTION

### Technical Problem

Treatments using EGFR inhibitors are in the process of being established for lung cancer patients having major EGFR mutations. In contrast, the presence of patients who are resistant to osimertinib treatment has also been known. It is reported that some of the causes thereof are EGFR gene mutations (NPL 4). It has been confirmed that, for example, lung cancer having, in addition to exon 19 deletion mutation or L858R mutation, both of which are de novo mutations, and T790M mutation, which is acquired resistance mutation, point mutation in which leucine encoded by codon 718 in exon 18 is substituted with an arbitrary amino acid (hereinafter also referred to as “L718X mutation”) or point mutation in which leucine encoded by codon 792 in exon 20 is substituted with an arbitrary amino acid (hereinafter also referred to as “L792X mutation”), is resistant to osimertinib treatment. It is proposed that amino acid substitution caused by L718X mutation or L792X mutation induces steric hindrance and decrease in hydrophobic binding, thereby preventing EGFR binding to osimertinib (NPL 4). Accordingly, there is a demand for the development of inhibitors having high inhibitory activity against EGFR having composite mutation of de novo active mutation, T790M acquired resistance mutation, and L718X mutation or L792X mutation.

Therefore, the development of drugs having high inhibitory activity against EGFR having composite mutation containing L718X or L792X is assumed to make it possible to exhibit antitumor effects against lung cancer that is resistant to osimertinib treatment, and is expected to contribute to the life prolongation and QOL improvement of mutant EGFR-positive cancer patients for whom no therapy has been established. Further, a drug having high inhibitory activity with respect to T790M, which is acquired resistance mutation against treatments using EGFR inhibitors, is expected to reduce expression frequency of acquired resistance during the treatments using EGFR inhibitors against exon 19 or exon 21 mutant EGFR, which is de novo mutation; and is therefore expected to contribute to the life prolongation of cancer patients.

Under such circumstances, an object of the present invention is to provide an inhibitor that has high inhibitory activity against L718 and L792 mutant EGFR, which is an osimertinib treatment-resistant mutation, and for which the therapeutic effects of the previously known EGFR inhibitors are insufficient.

### Solution to Problem

The inventors of the present invention conducted extensive research, and found that EGFR inhibitors conventionally introduced into treatment have poor inhibitory activity against composite mutant EGFR containing L718 and L792 mutations, in addition to active mutation and T790M acquired resistance mutation. Further, the inventors conducted extensive research starting from comparison of the cocrystal structures of compounds, and also found that the compound of the present invention exhibits excellent inhibitory activity against EGFR having L718 and L792 mutations, and further exhibits excellent inhibitory activity against the above composite mutant EGFR. With this finding, the inventors have accomplished the present invention.

The present invention encompasses the following embodiments.

### Item 1.

An antitumor agent for treating a malignant tumor patient expressing EGFR having at least one mutation selected from the group consisting of L718X mutation in exon 18 and L792X mutation in exon 20, wherein X represents an arbitrary amino-acid residue, the antitumor agent comprising (S)-N-(4-amino-6-methyl-5-(quinolin-3-yl)-8,9-dihydropyrimido[5,4-b]indolizin-8-yl)acrylamide (Compound (A)) or a salt thereof.

### Item 2.

The antitumor agent according to Item 1, wherein the EGFR further has at least one mutation selected from the group consisting of exon 19 deletion mutation, L858R, L861Q, G719X, E709X, and exon 20 insertion mutation.

### Item 3.

The antitumor agent according to Item 2, wherein the EGFR further has T790M mutation.

### Item 4.

The antitumor agent according to any one of Items 1 to 3, wherein the L718X mutation is L718Q mutation.

### Item 5.

The antitumor agent according to any one of Items 1 to 4, wherein the L792X mutation is L792H, L792F, or L792Y.

### Item 6.

A method for treating a malignant tumor patient, comprising the step of administering (S)-N-(4-amino-6-methyl-5-(quinolin-3-yl)-8,9-dihydropyrimido[5,4-b]indolizin-8-yl)acrylamide or a salt thereof to a malignant tumor patient expressing EGFR having at least one mutation selected from the group consisting of L718X mutation in exon 18 and L792X mutation in exon 20.

### Item 7.

(S)-N-(4-amino-6-methyl-5-(quinolin-3-yl)-8,9-dihydropyrimido[5,4-b]indolizin-8-yl)acrylamide or a salt thereof for treating a malignant tumor patient expressing EGFR having at least one mutation selected from the group consisting of L718X mutation in exon 18 and L792X mutation in exon 20.

### Item 8.

Use of (S)-N-(4-amino-6-methyl-5-(quinolin-3-yl)-8,9-dihydropyrimido[5,4-b]indolizin-8-yl)acrylamide or a salt thereof for treating a malignant tumor patient expressing EGFR having at least one mutation selected from the group consisting of L718X mutation in exon 18 and L792X mutation in exon 20.

### Item 9.

Use of (S)-N-(4-amino-6-methyl-5-(quinolin-3-yl)-8,9-dihydropyrimido[5,4-b]indolizin-8-yl)acrylamide or a salt thereof for the production of a pharmaceutical agent for treating a malignant tumor patient expressing EGFR having at least one mutation selected from the group consisting of L718X mutation in exon 18 and L792X mutation in exon 20.

### Item 10.

A pharmaceutical composition comprising (S)-N-(4-amino-6-methyl-5-(quinolin-3-yl)-8,9-dihydropyrimido[5,4-b]indolizin-8-yl)acrylamide or a salt thereof and a pharmaceutically acceptable carrier, for treating a malignant tumor patient expressing EGFR having at least one mutation selected from the group consisting of L718X mutation in exon 18 and L792X mutation in exon 20, wherein X represents an arbitrary amino-acid residue.

### Item 11.

A method for predicting therapeutic effects of chemotherapy using an antitumor agent comprising, as an active ingredient, (S)-N-(4-amino-6-methyl-5-(quinolin-3-yl)-8,9-dihydropyrimido[5,4-b]indolizin-8-yl)acrylamide or a salt thereof in a malignant tumor patient, the method comprising steps (1) and (2) below:
(1) a step of detecting the presence or absence of mutation of EGFR gene contained in a biological sample obtained from the patient; and
(2) a step of predicting that the chemotherapy is highly likely to exhibit sufficient therapeutic effects with respect to the patient when the results of the detection in step (1) find that the EGFR gene has at least one mutation selected from the group consisting of L718X mutation in exon 18 and L792X mutation in exon 20, wherein X represents an arbitrary amino-acid residue.

### Item 12.

A method for treating a malignant tumor patient, comprising steps (1) to (3) below:
(1) a step of detecting the presence or absence of mutation of EGFR gene contained in a biological sample obtained from the patient;
(2) a step of predicting that chemotherapy using an antitumor agent comprising, as an active ingredient, (S)-N-(4-amino-6-methyl-5-(quinolin-3-yl)-8,9-dihydropyrimido[5,4-b]indolizin-8-yl)acrylamide or a salt thereof is highly likely to exhibit sufficient therapeutic effects with respect to the patient when the results of the detection in step (1) find that the EGFR gene has at least one mutation selected from the group consisting of L718X mutation in exon 18 and L792X mutation in exon 20, wherein X represents an arbitrary amino-acid residue; and
(3) a step of administering (S)-N-(4-amino-6-methyl-5-(quinolin-3-yl)-8,9-dihydropyrimido[5,4-b]indolizin-8-yl)acrylamide or a salt thereof to a malignant tumor patient who has been predicted highly likely to sufficiently respond to the chemotherapy using an antitumor agent comprising, as an active ingredient, (S)-N-(4-amino-6-methyl-5-(quinolin-3-yl)-8,9-dihydropyrimido[5,4-b]indolizin-8-yl)acrylamide or a salt thereof, in step (2).

### Advantageous Effects of Invention

The antitumor agent of the present invention exerts high inhibitory activity against EGFR having L718 and L792 mutations. Therefore, the antitumor agent of the present invention is useful in view of providing an antitumor agent that exerts superior therapeutic effects for a malignant tumor patient expressing EGFR having L718 and L792 mutations.

The present invention is also useful in terms of providing a method for treating a malignant tumor patient expressing EGFR having L718 and L792 mutations.

The EGFR inhibitory activity of conventional EGFR inhibitors was remarkably reduced under the presence of T790M mutation, which is acquired resistance mutation in the exon 20 region, or under the presence of L718 and L792 mutations. Thus, it was difficult to exert sufficient therapeutic effects. In contrast, the antitumor agent of the present invention has high inhibitory activity against EGFR having L718 and/or L792 mutation, as well as having active mutation, such as Ex19del or L858R mutation; thus, the antitumor agent of the present invention can exert superior therapeutic effects for a malignant tumor patient expressing EGFR having these complex mutations. The antitumor agent of the present invention has high inhibitory activity against EGFR having L718 and/or L792 mutation, even though it has T790M mutation in addition to the above mutations; thus, the antitumor agent of the present invention can exert superior therapeutic effects for a malignant tumor patient expressing EGFR complexly having these mutations.

Further, the antitumor agent of the present invention is also useful in terms of reducing expression frequency of acquired resistance during the treatments using EGFR inhibitors against exon 18 or exon 21 mutant EGFR, which is de novo mutation, because of its high inhibitory activity against exon 18 and exon 20 treatment-resistant mutant EGFR even under the presence of T790M mutation, which is acquired resistance mutation in the exon 20 region.

### BRIEF DESCRIPTION OF DRAWINGS

FIG. 1 illustrates the evaluation results of phosphorylation (inhibitory activity) in a mutant EGFR forced expression system using HEK293 cells.

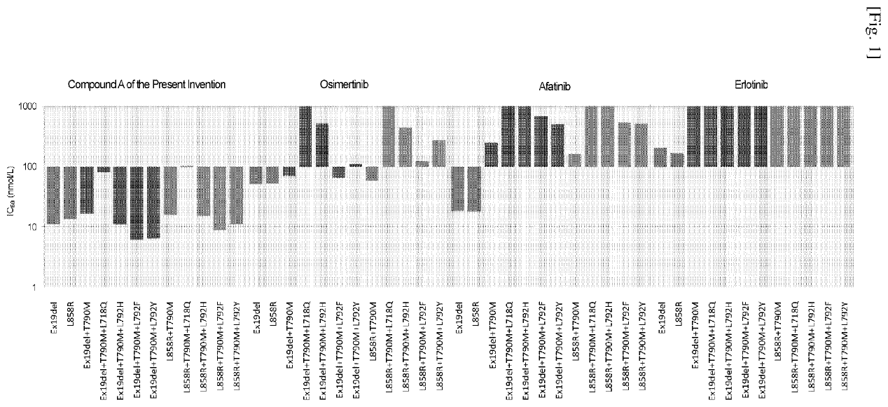

### DESCRIPTION OF EMBODIMENTS

Preferable examples of various definitions in the scope of the present invention used in this specification are explained below in detail.

In this specification, “EGFR” refers to a human epidermal growth factor receptor protein, and is also referred to as ErbB-1 or HER1.

In this specification, “wild-type EGFR” refers to EGFR free of somatic mutation, which is a protein comprising the amino acid sequence represented by SEQ ID NO: 1 (GenBank accession number: NP_005219.2).

In this specification, “exon 18” refers to 688-728 region in the amino acid sequence of wild-type EGFR (SEQ ID NO: 1).

In this specification, “exon 18 treatment-resistant mutation” refers to point mutation or deletion mutation in amino acid in the exon 18 region of wild-type EGFR (SEQ ID NO: 1). Preferable exon 18 treatment-resistant mutation is point mutation with 1 amino acid substitution in the exon 18 region. More preferably, the exon 18 treatment-resistant mutation is L718X (X represents, among amino acids constituting a protein encoded by genetic information, an arbitrary amino-acid residue other than leucine), which is point mutation in which leucine encoded by codon 718 of exon 18 is substituted with an arbitrary amino acid. More specifically, preferable examples of L718X include L718Q, which is point mutation in which leucine encoded by codon 718 in the exon 18 region is substituted with glutamine; and L718V, which is point mutation in which leucine encoded by codon 718 is substituted with valine.

In this specification, “exon 18 active mutation” refers to point mutation or deletion mutation in amino acid in the exon 18 region of wild-type EGFR (SEQ ID NO: 1). Preferable exon 18 active mutation is point mutation with 1 amino acid substitution in the exon 18 region. More preferably, the exon 18 active mutation is E709X, which is point mutation in which glutamic acid encoded by codon 709 of exon 18 is substituted with an arbitrary amino acid; or G719X, which is point mutation in which glycine encoded by codon 719 of exon 18 is substituted with an arbitrary amino acid. More specifically, preferable examples of E709X include E709K, which is point mutation in which glutamic acid encoded by codon 709 in the exon 18 region is substituted with lysine; and E709A, which is point mutation in which glutamic acid encoded by codon 709 in the exon 18 region is substituted with alanine. Preferable examples of G719X include G719A, which is point mutation in which glycine encoded by codon 719 in the exon 18 region is substituted with alanine; G719S, which is point mutation in which glycine encoded by codon 719 in the exon 18 region is substituted with serine; and G719C, which is point mutation in which glycine encoded by codon 719 in the exon 18 region is substituted with cysteine.

In the present invention, “exon 20” refers to 824-875 region in the amino acid sequence of wild-type EGFR (SEQ ID NO: 1).

In this specification, “exon 20 treatment-resistant mutation” refers to point mutation in amino acid in the exon 20 region of wild-type EGFR (SEQ ID NO: 1). Preferable exon 20 treatment-resistant mutation is point mutation with 1 amino acid substitution in the exon 20 region. More preferably, the exon 20 treatment-resistant mutation is L792X (X represents, among amino acids constituting a protein encoded by genetic information, an arbitrary amino-acid residue other than leucine), which is point mutation in which leucine encoded by codon 792 in the exon 20 region is substituted with an arbitrary amino acid. Specific examples include L792H, which is point mutation in which leucine encoded by codon 792 in the exon 20 region is substituted with histidine; L792F, which is point mutation in which leucine encoded by codon 792 is substituted with phenylalanine; L792Y, which is point mutation in which leucine encoded by codon 792 is substituted with tyrosine; L792R, which is point mutation in which leucine encoded by codon 792 is substituted with arginine; L792V, which is point mutation in which leucine encoded by codon 792 is substituted with valine; and L792P, which is point mutation in which leucine encoded by codon 792 is substituted with proline. Among these, L792F, L792H, and L792Y are preferable.

In this specification, “exon 20 insertion mutation” refers to mutation with insertion of one or more (preferably 1 to 7, more preferably 1 to 4) amino acids into the exon 20 region of EGFR (761 to 823 amino acid sequence in SEQ ID NO: 1). Preferable examples include mutation with insertion of amino acid sequence FQEA (phenylalanine, glutamine, glutamic acid, and alanine sequentially from the N-terminal side) between 763 alanine and 764 tyrosine in the exon 20 region (A763_Y764insFQEA), mutation with insertion of amino acid sequence ASV (alanine, serine, and valine sequentially from the N-terminal side) between 769 valine and 770 asparagic acid in the exon 20 region (V769_D770insASV), mutation with insertion of amino acid sequence SVD (serine, valine, and asparagic acid sequentially from the N-terminal side) between 770 asparagic acid and 771 asparagine in the exon 20 region (D770_N771insSVD), mutation with insertion of amino acid sequence NPG (asparagine, proline, and glycine sequentially from the N-terminal side) between 770 asparagic acid and 771 asparagine in the exon 20 region (D770_N771insNPG), mutation with insertion of amino acid G (glycine) between 770 asparagic acid and 771 asparagine in the exon 20 region (D770_N771insG), mutation with insertion of amino acid sequence GY (glycine and tyrosine sequentially from the N-terminal side) after deletion of 770 asparagic acid in the exon 20 region (D770>GY), mutation with insertion of amino acid N (asparagine) between 771 asparagine and 772 proline in the exon 20 region (N771_P772insN), mutation with insertion of amino acid sequence PR (proline and arginine sequentially from the N-terminal side) between 772 proline and 773 histidine in the exon 20 region (P772_R773insPR), mutation with insertion of amino acid sequence NPH (asparagine, proline, and histidine sequentially from the N-terminal side) between 773 histidine and 774 valine in the exon 20 region (H773_V774insNPH), mutation with insertion of amino acid sequence PH (proline and histidine sequentially from the N-terminal side) between 773 histidine and 774 valine in the exon 20 region (H773_V774insPH), mutation with insertion of amino acid sequence AH (alanine and histidine sequentially from the N-terminal side) between 773 histidine and 774 valine in the exon 20 region (H773_V774insAH), mutation with insertion of amino acid H (histidine) between 773 histidine and 774 valine in the exon 20 region (H773_V774insH), mutation with insertion of amino acid sequence HV (histidine and valine sequentially from the N-terminal side) between 774 valine and 775 cysteine in the exon 20 region (V774_C774insHV), mutation with insertion of amino acid sequence EAFQ (glutamic acid, alanine, phenylalanine, and glutamine sequentially from the N-terminal side) between 761 alanine and 762 glutamic acid in the exon 20 region (A761_E762insEAFQ), and the like. More preferable examples include mutation with insertion of amino acid sequence ASV (alanine, serine, and valine sequentially from the N-terminal side) between 769 valine and 770 asparagic acid in the exon 20 region (V769_D770insASV), mutation with insertion of amino acid sequence SVD (serine, valine, and asparagic acid sequentially from the N-terminal side) between 770 asparagic acid and 771 asparagine in the exon 20 region (D770_N771insSVD), mutation with insertion of amino acid G (glycine) between 770 asparagic acid and 771 asparagine in the exon 20 region (D770_N771insG), mutation with insertion of amino acid sequence NPH (asparagine, proline, and histidine sequentially from the N-terminal side) between 773 histidine and 774 valine in the exon 20 region (H773_V774insNPH), and mutation with insertion of amino acid sequence PH (proline and histidine sequentially from the N-terminal side) between 773 histidine and 774 valine in the exon 20 region (H773_V774insPH). Particularly preferable examples include mutation with insertion of amino acid sequence FQEA (phenylalanine, glutamine, glutamic acid, and alanine sequentially from the N-terminal side) between 763 alanine and 764 tyrosine in the exon 20 region (A763_Y764insFQEA), mutation with insertion of amino acid sequence ASV (alanine, serine, and valine sequentially from the N-terminal side) between 769 valine and 770 asparagic acid in the exon 20 region (V769_D770insASV), mutation with insertion of amino acid sequence SVD (serine, valine, and asparagic acid sequentially from the N-terminal side) between 770 asparagic acid and 771 asparagine in the exon 20 region (D770_N771insSVD), mutation with insertion of amino acid G (glycine) between 770 asparagic acid and 771 asparagine in the exon 20 region (D770_N771insG), mutation with insertion of amino acid sequence NPH (asparagine, proline, and histidine sequentially from the N-terminal side) between 773 histidine and 774 valine in the exon 20 region (H773_V774inchsNPH), mutation with insertion of amino acid sequence PH (proline and histidine sequentially from the N-terminal side) between 773 histidine and 774 valine in the exon 20 region (H773_V774insPH), and the like.

In the present invention, “exon 21” refers to 824-875 region in the amino acid sequence of wild-type EGFR (SEQ ID NO: 1).

In this specification, “exon 21 active mutation” refers to point mutation in amino acid in the exon 21 region of wild-type EGFR (SEQ ID NO: 1). Preferable exon 21 active mutation is point mutation with 1 amino acid substitution in the exon 21 region. More preferably, the exon 21 active mutation is L858X, which is point mutation in which leucine encoded by codon 858 in the exon 21 region is substituted with an arbitrary amino acid; or L861X, which is point mutation in which leucine encoded by codon 861 in the exon 21 region is substituted with an arbitrary amino acid (X represents, among amino acids constituting a protein encoded by genetic information, an arbitrary amino-acid residue other than leucine). More specifically, preferable examples include L858R, which is point mutation in which leucine encoded by codon 858 in the exon 21 region is mutated to arginine; and L861Q, which is point mutation in which leucine encoded by codon 861 in the exon 21 region is substituted with glutamine.

In the present invention, “exon 18 and/or exon 20 treatment-resistant mutation” encompasses “exon 18 treatment-resistant mutation,” “exon 20 treatment-resistant mutation,” and “exon 18 and exon 20 treatment-resistant mutation.”

In the present invention, “point mutation” refers to mutation causing substitution, insertion, or deletion of one or more (e.g., about 1 to 10, preferably about 1 to 5, more preferably about 1, 2, or 3)amino-acid residues; and may include in-frame insertion and/or deletion mutation as nucleic acid.

“EGFR having exon 18 and/or exon 20 treatment-resistant mutation” encompasses “EGFR having exon 18 treatment-resistant mutation,” “EGFR having exon 20 treatment-resistant mutation,” and “EGFR having exon 18 and exon 21 treatment-resistant mutation.”

In this specification, “EGFR having exon 18 treatment-resistant mutation” refers to EGFR having at least the above L718X mutation in exon 18 as the exon 18 treatment-resistant mutation. The EGFR may further have an exon 18 mutation other than L718X, but preferably has a single L718X mutation as the exon 18 treatment-resistant mutation. Moreover, the EGFR may have a mutation other than exon 18 treatment-resistant mutation (e.g., exon 19 deletion mutation, L858R mutation, and L790M mutation).

In this specification, the “EGFR having exon 20 treatment-resistant mutation” refers to EGFR having at least the above L792X mutation in exon 20 as the exon 20 treatment-resistant mutation. The EGFR may have a mutation other than L792X mutation, but preferably has a single L718X mutation as the exon treatment-resistant 20 mutation. Moreover, the EGFR may have a mutation other than exon 20 treatment-resistant mutation (e.g., exon 19 deletion mutation, L858R mutation, and L790M mutation).

In this specification, “exon 19” refers to 729-823 region in the amino acid sequence of wild-type EGFR (SEQ ID NO: 1).

In this specification, “exon 19 deletion mutation” refers to mutation with deletion of one or more amino acids in the exon 19 region of wild-type EGFR (SEQ ID NO: 1). In addition to deletion in this region, mutation with insertion of one or more arbitrary amino acids is also included. Examples of exon 19 deletion mutation include mutation with deletion of 5 amino acids from 746 glutamic acid to 750 alanine in the exon 19 region (Del E746-A750), mutation with insertion of serine after deletion of 7 amino acids from 747 leucine to 753 proline in the exon 19 region (Del 747-P753insS), mutation with deletion of 5 amino acids from 747 leucine to 751 threonine in the exon 19 region (Del L747-T751), mutation with insertion of proline after deletion of 4 amino acids from 747 leucine to 750 alanine in the exon 19 region (Del 747-A750insP), and the like. Preferable examples include mutation with deletion of 5 amino acids from 746 glutamic acid to 750 alanine in the exon 19 region (Del E746-A750).

Moreover, the EGFR having exon 18 and/or exon 20 treatment-resistant mutation may further have at least one mutation selected from the group consisting of exon 19 deletion mutation, L858R, L861Q, G719X, E709X, and exon 20 insertion mutation. Specific examples include EGFR having L718X mutation in the exon 18 region further having exon 19 deletion mutation, EGFR having L792X mutation in the exon 20 region further having exon 19 deletion mutation, EGFR having L718X mutation in the exon 18 region further having L858R, EGFR having L792X mutation in the exon 20 region further having L858R, EGFR having L718X mutation in the exon 18 region further having L861Q, EGFR having L792X mutation in the exon 20 region further having L861Q, EGFR having L718X mutation in the exon 18 region further having G719X, EGFR having L792X mutation in the exon 20 region further having G719X, EGFR having L718X mutation in the exon 18 region further having E709X, EGFR having L792X mutation in the exon 20 region further having E709X, EGFR having L718X mutation in the exon 18 region further having exon 20 insertion mutation, and EGFR having L792X mutation in the exon 20 region further having exon 20 insertion mutation. Preferable among these are EGFR having L718X mutation in the exon 18 region further having exon 19 deletion mutation, EGFR having L792X mutation in the exon 20 region further having exon 19 deletion mutation, EGFR having L718X mutation in the exon 18 region further having L858R, and EGFR having L792X mutation in the exon 20 region further having L858R.

Further, the EGFR having exon 18 and/or exon 20 treatment-resistant mutation may further have T790M mutation, in addition to the above exon 19 deletion mutation, L858R, L861Q, G719X, E709X, and exon 20 insertion mutation. T790M is acquired resistance mutation in the exon 20 region. T790M is known to be generated by the use of existing EGFR inhibitors. The acquisition of T790M often decreases the effects of existing drugs with respect to malignant tumor patients.

In the present invention, examples include EGFR having L718X mutation in the exon 18 region having exon 19 deletion mutation further having T790M mutation, EGFR having L792X mutation in the exon 20 region having exon 19 deletion mutation further having T790M mutation, EGFR having L718X mutation in the exon 18 region having L858R further having T790M mutation, EGFR having L792X mutation in the exon 20 region having L858R further having T790M mutation, EGFR having L718X mutation in the exon 18 region having L861Q further having T790M mutation, EGFR having L792X mutation in the exon 20 region having L861Q further having T790M mutation, EGFR having L718X mutation in the exon 18 region having G719X further having T790M mutation, EGFR having L792X mutation in the exon 20 region having G719X further having T790M mutation, EGFR having L718X mutation in the exon 18 region having E709X further having T790M mutation, EGFR having L792X mutation in the exon 20 region having E709X further having T790M mutation, EGFR having L718X mutation in the exon 18 region having exon 20 insertion mutation further having T790M mutation, and EGFR having L792X mutation in the exon 20 region having exon 20 insertion mutation further having T790M mutation. Preferable among these are EGFR having L718X mutation in the exon 18 region having exon 19 deletion mutation further having T790M mutation, EGFR having L792X mutation in the exon 20 region having exon 19 deletion mutation further having T790M mutation, EGFR having L718X mutation in the exon 18 region having L858R further having T790M mutation, and EGFR having L792X mutation in the exon 20 region having L858R further having T790M mutation.

Among the EGFRs having the above composite mutations, EGFR having L718Q mutation in the exon 18 region, EGFR having L792F mutation in the exon 20 region, EGFR having L792H mutation, and EGFR having L792Y mutation are particularly preferable.

In the present invention, the method for detecting mutations in EGFR expressed by a malignant tumor patient is not particularly limited insofar as the method is capable of detecting the mutations, and any known detection methods may be used.

The sample used in the detection of EGFR mutation is not particularly limited as long as the sample is a biological sample isolated from a malignant tumor patient, in particular, a sample obtained from a malignant tumor patient and containing malignant tumor cells.

Examples of biological samples include body fluids (e.g., blood, urine, etc.), tissues, the extracts thereof, and the cultures of obtained tissues. The method for obtaining a biological sample can be suitably selected depending on the type of biological sample.

The biological sample is prepared by being appropriately treated according to the measurement method. Further, the reagent comprising a primer or probe used for the detection may be prepared by a conventional method according to the measurement method therefor.

In one embodiment of the present invention, a step of detecting the presence of the mutation of the present invention in EGFR expressed by a malignant tumor patient may be performed before the administration of an antitumor agent to the malignant tumor patient.

A malignant tumor may include two or more different kinds of malignant tumor cells.

Further, two or more malignant tumors may be generated in a single patient. Therefore, a single patient may have different mutations of EGFR (for example, the exon 18 mutation is L718Q and L718V exon 18 mutation; and the exon 20 mutation is L792F, L792H, L792Y, L792R, L792V, and L792P exon 20 mutation; however, there is no limitation thereto) at the same time.

The antitumor agent of the present invention comprises, as an active ingredient, (S)-N-(4-amino-6-methyl-5-(quinolin-3-yl)-8,9-dihydropyrimido[5,4-b]indolizin-8-yl)acrylamide (Compound (A)) or a salt thereof. Compound (A) is represented by the following chemical formula.

The method for producing the compound of the present invention is explained below.

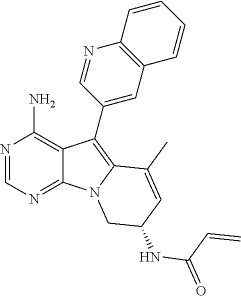 [SMILES: C=CC(=O)N[C@H]1C=C(C)c2c(-c3cnc4ccccc4c3)c3c(N)ncnc3n2C1]

Compound A of the present invention may be produced, for example, through the production method disclosed in WO2015/025936A1, the methods described in the Examples, and the like. However, the production method of the compound of the present invention is not limited to these reaction examples.

When Compound A of the present invention has isomers such as optical isomers, stereoisomers, and tautomers, any of the isomers and mixtures thereof are included within the scope of the compound of the present invention, unless otherwise specified. For example, when the compound of the present invention has optical isomers, racemic mixtures and the optical isomers separated from a racemic mixture are also included within the scope of the compound of the present invention, unless otherwise specified.

The salts of Compound A refer to any pharmaceutically acceptable salts; examples include base addition salts and acid addition salts.

Examples of base addition salts include alkali metal salts such as sodium salts and potassium salts; alkaline earth metal salts such as calcium salts and magnesium salts; ammonium salts; and organic amine salts such as trimethylamine salts, triethylamine salts, dicyclohexylamine salts, ethanolamine salts, diethanolamine salts, triethanolamine salts, procaine salts, and N,N′-dibenzylethylenediamine salts.

Examples of acid addition salts include inorganic acid salts such as hydrochlorides, sulfates, nitrates, phosphates, and perchlorates; organic acid salts such as acetates, formates, maleates, fumarates, tartrates, citrates, ascorbates, and trifluoroacetates; and sulfonates such as methanesulfonates, isethionates, benzenesulfonates, and p-toluenesulfonates.

The compound of the present invention or salts thereof also encompass prodrugs thereof. A prodrug refers to a compound that can be converted to the compound of the present invention or a salt thereof through a reaction with an enzyme, gastric acid, or the like, under physiological conditions in vivo, i.e., a compound that can be converted to the compound of the present invention or a salt thereof by enzymatic oxidation, reduction, hydrolysis, or the like; or a compound that can be converted to the compound of the present invention or a salt thereof by hydrolysis or the like with gastric acid or the like. Further, the prodrug may be a compound that can be converted to the compound of the present invention or a salt thereof under physiological conditions, such as those described in “Iyakuhin no Kaihatsu [Development of Pharmaceuticals],” Vol. 7, Molecular Design, published in 1990 by Hirokawa Shoten Co., pp. 163-198.

### Description of Diseases

Specific examples of tumors targeted in the present invention include, but are not particularly limited to, head and neck cancer, gastrointestinal cancer (esophageal cancer, stomach cancer, duodenal cancer, liver cancer, biliary cancer (e.g., gallbladder and bile duct cancer), pancreatic cancer, colorectal cancer (e.g., colon cancer and rectal cancer), etc.), lung cancer (e.g., non-small-cell lung cancer, small-cell lung cancer, and mesothelioma), breast cancer, genital cancer (ovarian cancer, uterine cancer (e.g., cervical cancer and endometrial cancer), etc.), urological cancer (e.g., kidney cancer, bladder cancer, prostate cancer, and testicular tumor), hematopoietic tumor (e.g., leukemia, malignant lymphoma, and multiple myeloma), osteosarcoma, soft-tissue sarcoma, skin cancer, brain tumor, and the like.

Preferable examples include lung cancer, breast cancer, head and neck cancer, brain tumor, uterine cancer, gastrointestinal cancer, hematopoietic tumor, and skin cancer. Lung cancer is particularly preferable.

When the compound of the present invention or a salt thereof is used as a pharmaceutical agent, a pharmaceutical carrier can be added, if required, thereby forming a suitable dosage form according to prevention and treatment purposes. Examples of the dosage form include oral preparations, injections, suppositories, ointments, patches, and the like. Oral preparations are preferable. Such dosage forms can be formed by methods conventionally known to persons skilled in the art.

As the pharmaceutically acceptable carrier, various conventional organic or inorganic carrier materials used as preparation materials may be blended as an excipient, binder, disintegrant, lubricant, or colorant in solid preparations; or as a solvent, solubilizing agent, suspending agent, isotonizing agent, buffer, or soothing agent in liquid preparations. Moreover, pharmaceutical preparation additives, such as antiseptics, antioxidants, colorants, sweeteners, and stabilizers, may also be used, if required.

Oral solid preparations are prepared as follows. After an excipient is added optionally with an excipient, binder, disintegrant, lubricant, colorant, taste-masking or flavoring agent, etc., to the compound of the present invention, the resulting mixture is formulated into tablets, coated tablets, granules, powders, capsules, or the like by ordinary methods.

Examples of excipients include lactose, sucrose, D-mannitol, glucose, starch, calcium carbonate, kaolin, microcrystalline cellulose, and silicic acid anhydride. Examples of binders include water, ethanol, 1-propanol, 2-propanol, simple syrup, liquid glucose, liquid α-starch, liquid gelatin, D-mannitol, carboxymethyl cellulose, hydroxypropyl cellulose, hydroxypropyl starch, methyl cellulose, ethyl cellulose, shellac, calcium phosphate, polyvinylpyrrolidone, and the like. Examples of disintegrators include dry starch, sodium alginate, powdered agar, sodium hydrogen carbonate, calcium carbonate, sodium lauryl sulfate, stearic acid monoglyceride, lactose, and the like. Examples of lubricants include purified talc, sodium stearate, magnesium stearate, borax, polyethylene glycol, and the like. Examples of colorants include titanium oxide, iron oxide, and the like. Examples of taste-masking or flavoring agents include sucrose, bitter orange peel, citric acid, tartaric acid, and the like.

When a liquid preparation for oral administration is prepared, a taste-masking agent, a buffer, a stabilizer, a flavoring agent, and the like may be added to the compound of the present invention; and the resulting mixture may be formulated into an oral liquid preparation, syrup, elixir, etc., according to an ordinary method.

When an injection agent is prepared, a pH regulator, a buffer, a stabilizer, an isotonizing agent, a local anesthetic, and the like, may be added to the compound of the present invention; and the mixture may be formulated into a subcutaneous, intramuscular, or intravenous injection according to an ordinary method.

Examples of the pH adjuster and the buffer used herein include sodium citrate, sodium acetate, and sodium phosphate. Examples of the stabilizer include sodium pyrosulfite, EDTA, thioglycolic acid, and thiolactic acid. Examples of the local anesthetic include procaine hydrochloride and lidocaine hydrochloride. Examples of the isotonizing agent include sodium chloride, glucose, D-mannitol, and glycerol.

When a suppository is prepared, pharmaceutically acceptable carriers known in the related field, such as polyethylene glycol, lanolin, cacao butter, and fatty acid triglyceride; and, as necessary, surfactants such as Tween 80 (registered trademark), may be added to Compound A, and the resulting mixture may be formulated into a suppository according to an ordinary method.

When an ointment is prepared, a commonly used base, stabilizer, wetting agent, preservative, and the like, may be blended into Compound A, as necessary; and the obtained mixture is mixed and formulated into an ointment according to an ordinary method.

Examples of the base include liquid paraffin, white petrolatum, white beeswax, octyl dodecyl alcohol, and paraffin.

Examples of the preservative include methyl paraoxybenzoate, ethyl paraoxybenzoate, and propyl paraoxybenzoate.

When a patch is prepared, the above-described ointment, cream, gel, paste, or the like, may be applied to an ordinary substrate according to an ordinary method.

Examples of substrates include woven fabrics or non-woven fabrics comprising cotton, staple fibers, or chemical fibers; and films or foam sheets of soft vinyl chloride, polyethylene, polyurethane, etc., may also be used.

The amount of Compound A to be incorporated in each of such dosage unit forms depends on the condition of the patient to whom the compound is administered, the dosage form thereof, etc. In general, in the case of an oral agent, the amount of the compound is preferably 0.05 to 1000 mg per dosage unit form. In the case of an injection, the amount of the compound is preferably 0.01 to 500 mg per dosage unit form; and in the case of a suppository, the amount of the compound is preferably 1 to 1000 mg per dosage unit form.

Further, the daily dose of the medicine in such a dosage form depends on the condition, body weight, age, sex, etc., of the patient, and cannot be generalized. Usually, the daily dose for an adult (body weight: 50 kg) of the compound of the present invention may generally be 0.05 to 5000 mg, and preferably 0.1 to 1000 mg; and is preferably administered in one dose, or in two to three divided doses, per day.

The present invention also provides a method for treating a malignant tumor patient, comprising the step of administering Compound A or a salt thereof to a malignant tumor patient expressing EGFR having exon 18 and/or exon 20 treatment-resistant mutation.

The present invention also provides Compound A or a salt thereof for treating a malignant tumor patient expressing EGFR having exon 18 and/or exon 20 treatment-resistant mutation.

The present invention also provides use of Compound A or a salt thereof for treating a malignant tumor patient expressing EGFR having exon 18 and/or exon 20 treatment-resistant mutation.

The present invention also provides use of Compound A or a salt thereof for the production of a pharmaceutical agent for treating a malignant tumor patient expressing EGFR having exon 18 and/or exon 20 treatment-resistant mutation.

The present invention also provides a method for predicting therapeutic effects of chemotherapy using an antitumor agent comprising, as an active ingredient, Compound A or a salt thereof in a malignant tumor patient, the method comprising steps (1) and (2) below:
(1) a step of detecting the presence or absence of mutation of EGFR gene contained in a biological sample obtained from the patient; and
(2) a step of predicting that the chemotherapy is highly likely to exhibit sufficient therapeutic effects to the patient when the results of the detection in step (1) find that the EGFR gene has exon 18 and/or exon 20 treatment-resistant mutation.

The present invention also provides a method for treating a malignant tumor patient, comprising steps (1) to (3) below:
(1) a step of detecting the presence or absence of mutation of EGFR gene contained in a biological sample obtained from the patient; and
(2) a step of predicting that chemotherapy using an antitumor agent comprising, as an active ingredient, (S)-N-(4-amino-6-methyl-5-(quinolin-3-yl)-8,9-dihydropyrimido[5,4-b]indolizin-8-yl)acrylamide or a salt thereof is highly likely to exhibit sufficient therapeutic effects with respect to the patient when the results of the detection in step (1) find that the EGFR gene has exon 18 and/or exon 20 treatment-resistant mutation; and
(3) a step of administering (S)-N-(4-amino-6-methyl-5-(quinolin-3-yl)-8,9-dihydropyrimido[5,4-b]indolizin-8-yl)acrylamide or a salt thereof to a malignant tumor patient who has been predicted highly likely to sufficiently respond to the chemotherapy using an antitumor agent comprising, as an active ingredient, (S)-N-(4-amino-6-methyl-5-(quinolin-3-yl)-8,9-dihydropyrimido[5,4-b]indolizin-8-yl)acrylamide or a salt thereof, in step (2).

The base sequence of EGFR gene is publicly known. The GenBank accession number of the base sequence of cDNA is NM_005228.4.

The “therapeutic effects” can be evaluated by tumor shrinkage effects, relapse-suppressing effects, life-prolonging effects, and the like. The relapse-suppressing effects may be shown as the degree of the extension of non-relapse period or the degree of the improvement in relapse rate; and the life-prolonging effects may be shown as the degree of the entire survival time or the degree of the extension of the median of progression-free survival, or the like. The “sufficient therapeutic effects” of the chemotherapy using an antitumor agent comprising, as an active ingredient, Compound A or a salt thereof means, for example, that superior therapeutic effects are obtained by the administration of the antitumor agent comprising, as an active ingredient, Compound A or a salt thereof, such as extension of survival time, suppression of relapse, and the like, compared with non-administration.

### EXAMPLES

The following describes the present invention in more detail with reference to the following Test Example. However, the present invention is not limited to this Example (Test Example).

### Test Example 1

### In Vitro Drug Efficacy Test

Evaluation Results of Intracellular Phosphorylation in Mutant EGFR Forced Expression System Using HEK293 Cells (Inhibitory activity)

The intracellular target inhibitory activity of compounds was evaluated based on the following as an index: intracellular EGFR phosphorylation in a mutant EGFR forced expression system using Jump-In (trademark) Grip (trademark) HEK293 cells (Thermo Fisher Scientific Inc.) (hereinafter also referred to as “HEK293 cells”). The HEK293 cells were maintained in D-MEM with GlutaMAX (trademark)-I (high glucose) (Thermo Fisher Scientific Inc.) that contained 10% dialyzed FBS. The HEK293 cells were seeded in each well of a 96-well flat-bottom microplate such that the cell count per well was 10,000, and incubated in a 5% CO2 gas-containing incubator at 37° C. overnight. Then, a pcDNATM 6.2/V5-DEST vector encoding a human EGFR gene (Del E746-A750 (hereinafter also referred to as “Ex19del”), Ex19del+T790M (the symbol “+” indicates that both mutations are contained), Ex19del+T790M+L718Q, Ex19del+T790M+L792H, Ex19del+T790M+L792F, Ex19del+T790M+L792Y, L858R, L858R+T790M, L858R+T790M+L718Q, L858R+T790M+L792H, L858R+T790M+L792F, or L858R+T790M+L792Y) was introduced, together with Opti-MEM (trademark) I (Thermo Fisher Scientific Inc.), using a ViaFect (trademark) Transfection Reagent (Promega Corporation). The cells were incubated again in a 5% CO2 gas-containing incubator at 37° C. overnight. The following day, Compound A, erlotinib, afatinib, and osimertinib (erlotinib, afatinib, and osimertinib may each be hereinafter referred to as a “comparative compound”) were individually dissolved in DMSO, and diluted with DMSO or a medium. The solutions were then individually added to each well of the culture plate of the cells, and the cells were incubated in a 5% CO2 gas-containing incubator at 37° C. for 6 hours. After incubation, the cells were immobilized using 20% neutral buffered formalin (Wako Pure Chemical Industries, Ltd.), and blocked by an Odyssey (trademark) blocking buffer (PBS) (M&S TechnoSystems Inc.). The cells were then reacted with a primary antibody (EGFR Antibody Cocktail #AHR5062 (Thermo Fisher Scientific Inc.) and a Phospho-EGFR Receptor (Tyr1068) Antibody #2234L (CST)) diluted with an Odyssey (trademark) blocking buffer (PBS) to 1/200, and the cells were allowed to stand at 4° C. overnight. The following day, the cells were reacted with a secondary antibody (IRDye 800CW Goat aRabbit #926-32211 and IRDye 680RD Goat aMouse #926-68070 (M&S TechnoSystems Inc.)) diluted with an Odyssey (trademark) blocking buffer (PBS) to 1/800, and the cells were allowed to stand at room temperature for 1 hour. The fluorescence intensity (hereinafter also referred to as “FI”) was detected with an Odyssey (trademark) CLx Infrared Imaging System (LI-COR Bioscience) at a fluorescence wavelength of 800 nm and 700 nm.

The value obtained by subtracting the FI of a well without the primary antibody from the FI detected at a fluorescence wavelength of 800 nm or 700 nm was referred to as FI (800, EGFR)-Blank (for 800 nm) and FI (700, p-EGFR)-Blank (for 700 nm). The value obtained by dividing FI (700, p-EGFR)-Blank of each well by FI (800, EGFR)-Blank was determined to be FI (p-EGFR/EGFR). The phosphorylated EGFR rate was calculated using the following formula to determine the concentration of the test compounds at which EGFR was phosphorylated by 50% (IC50 (ΞM)). Table 1 illustrates the results.

$$Phosphorylated EGFR Rate ( \% ) = T / C \times 100$$

T: FI (p-EGFR/EGFR) of a well to which a test compound was added.
C: FT (p-EGFR/EGFR) of a well to which a test compound was not added.

As is clear from Table 1, Compound A exhibited high inhibitory activity against intracellular phosphorylation of composite mutant EGFR containing L718Q and L792X; and the activity was higher than that of erlotinib, afatinib, and osimertinib.

TABLE 1

| | Compound A of the | | | |
|---|---|---|---|---|
| | present invention | Osimertinib | Afatinib | Erlotinib |
| Ex19del | 11.2 ± 6.4 | 52.1 ± 23.6 | 18.5 ± 10.2 | 2016 |
| Ex19del + T790M | 16.9 ± 5.9 | 69.7 ± 15.6 | 250 ± 64 | >1000 |
| Ex19del + T790M + | 82.5 ± 9.0 | 5800 ± 2940 | >10000 | >10000 |
| L718Q | | | | |
| Ex19del + T790M + | 11.1 ± 5.6 | 513 ± 280 | 8190 ± 560 | >10000 |
| L792H | | | | |
| Ex19de1 + T790M + | 6.25 ± 1.83 | 65.7 ± 15.5 | 692 ± 325 | >10000 |
| L792F | | | | |
| Ex19del + T790M + | 6.54 ± 1.49 | 111 ± 38 | 508 ± 218 | >10000 |
| L792Y | | | | |
| L858R | 13.8 ± 13.8 | 53.3 ± 11.0 | 17.9 ± 3.0 | 167 ± 22 |
| L858R + T790M | 16.0 ± 2.6 | 59.0 ± 10.9 | 162 ± 38 | >1000 |
| L858R + T790M + | 104 ± 40 | 7880 ± 950 | 3670 ± 1690 | >10000 |
| L718Q | | | | |
| L858R + T790M + | 15.3 ± 5.0 | 441 ± 161 | 2190 ± 1310 | >10000 |
| L792H | | | | |
| L858R + T790M + | 9.07 ± 4.74 | 124 ± 40 | 538 ± 274 | >10000 |
| L792F | | | | |
| L858R + T790M + | 11.3 ± 5.8 | 273 ± 72 | 512 ± 269 | >10000 |
| L792Y | | | | |

1. A method for predicting therapeutic effects of chemotherapy using an antitumor agent comprising, as an active ingredient, (S)-N-(4-amino-6-methyl-5-(quinolin-3-yl)-8,9-dihydropyrimido[5,4-b]indolizin-8-yl)acrylamide or a salt thereof in a malignant tumor patient,
the method comprising:
(1) detecting the presence or absence of a mutation of EGFR gene contained in a biological sample obtained from the patient; and
(2) predicting based on results of the detecting in (1) that the chemotherapy is highly likely to exhibit sufficient therapeutic effects with respect to the patient when the results of the detection in (1) show that the EGFR gene has at least one mutation selected from the group consisting of L718X mutation in exon 18 and L792X mutation in exon 20, wherein X represents an arbitrary amino-acid residue.

2. The method of claim 1, wherein the EGFR further has at least one mutation selected from the group consisting of exon 19 deletion mutations L858R, L861Q, G719X, E709X, and an exon 20 insertion mutation.

3. The method according to claim 2, wherein the EGFR further has a T790M mutation.

4. The method according to claim 1, wherein the L71 8X mutation is a L718Q mutation.

5. The method according to claim 1, wherein the L792X mutation is L792H mutation, L792F mutation, or L792Y mutation.

6. A method for treating a malignant tumor patient, the method comprising:
(1) detecting the presence or absence of a mutation of EGFR gene contained in a biological sample obtained from the malignant tumor patient;
(2) predicting based on results of the detecting in (1) that chemotherapy using an antitumor agent comprising, as an active ingredient, (S)-N-(4-amino-6-methyl-5-(quinolin-3-yl)-8,9-dihydropyrimido[5,4-b]indolizin-8-yl)acrylamide or a salt thereof is highly likely to exhibit sufficient therapeutic effects with respect to the malignant tumor patient when the results of the detection in (1) show that the EGFR gene has at least one mutation selected from the group consisting of L718X mutation in exon 18 and L792X mutation in exon 20, wherein X represents an arbitrary amino-acid residue; and
(3) administering (S)-N-(4-amino-6-methyl-5-(quinolin-3-yl)-8,9-dihydropyrimido[5,4-b]indolizin-8-yl)acrylamide or a salt thereof to a malignant tumor patient who has been predicted highly likely to sufficiently respond to the chemotherapy using an antitumor agent comprising, as an active ingredient, (S)-N-(4-amino-6-methyl-5-(quinolin-3-yl)-8,9-dihydropyrimido[5,4-b]indolizin-8-yl)acrylamide or a salt thereof, in the predicting (2).

7. The method according to claim 6, wherein the (S)-N-(4-amino-6-methyl-5-(quinolin-3-yl)-8,9-dihydropyrimido[5,4-b]indolizin-8-yl)acrylamide or a salt thereof is administered to the malignant tumor patient in a form of a pharmaceutical composition further comprising a pharmaceutically acceptable carrier.

8. The method of claim 6, wherein the EGFR further has at least one mutation selected from the group consisting of exon 19 deletion mutations L858R, L861Q, G719X, E709X, and an exon 20 insertion mutation.

9. The method according to claim 8, wherein the EGFR further has a T790M mutation.

10. The method according to claim 6, wherein the L71 8X mutation is a L718Q mutation.

11. The method according to claim 6, wherein the L792X mutation is L792H mutation, L792F mutation, or L792Y mutation.

12. The method according to claim 6, wherein the patient has lung cancer.


SEQUENCE LISTING (1 sequence)

SEQ ID NO: 1 - protein, 1210 aa, Homo sapiens [sequence omitted]